(19) United States
(12) Patent Application Publication
(10) Pub. No.: US 2023/0116019 A1
(43) Pub. Date: Apr. 13, 2023

(54) NANOPARTICLES FOR EXPRESSION OF GENES OF INTEREST AND/OR REGULATION OF SIGNALING PATHWAYS

(71) Applicant: Andranik Andrew Aprikyan, Seattle, WA (US)

(72) Inventor: Andranik Andrew Aprikyan, Seattle, WA (US)

(73) Assignee: Stemgenics, Inc., Seattle, WA (US)

(21) Appl. No.: 17/758,620

(22) PCT Filed: Jan. 8, 2021
(86) PCT No.: PCT/US2021/012820

§ 371 Date: Jul. 11, 2022

Related U.S. Application Data
(60) Provisional application No. 62/960,626, filed on Jan. 13, 2020.
(60) Provisional application No. 62/959,790, filed on Jan. 10, 2020.

(51) Int. Cl. A61K 47/69; B82Y 5/00; A61K 47/62; C12N 15/113; C12N 15/87; A61K 47/54; A61K 48/00

(52) U.S. Cl. CPC A61K 47/6929; B82Y 5/00; A61K 47/62; C12N 15/1137; C12N 15/87; A61K 47/549; A61K 48/0025; C12N 2310/14; C12N 2320/32; C12N 2310/351

(57) ABSTRACT

The disclosure provides methods and compositions for delivering RNA constructs to cells for functional expression and/or activity. In some aspects, the disclosure provides a composition comprising a multi-functionalized nanoparticle. The multi-functionalized nanoparticles comprise a core functionalized with at least one RNA molecule, at least one cell penetrating peptide (CPP), and at least one positively charged moiety, each of which is independently attached to the core, optionally with linker moieties. In some embodiments, the RNA molecule is an uncapped mRNA molecule with the 5′ end attached to a linker moiety that is attached to the core. The multi-functionalized nanoparticle is substantially neutral, negatively or positively charged. The multi-functionalized nanoparticle can be used in methods of delivering and causing the expression of polypeptides of interest in a cell for various purposes, including vaccination, cancer treatment, extension of telomeres, modification of cellular signaling pathways, and the like.

### CROSS-REFERENCES TO RELATED APPLICATIONS

This application claims the benefit of U.S. Provisional Application No. 62/959,790, filed Jan. 10, 2020, and U.S. Provisional Application No. 62/960,626, filed Jan. 13, 2020, each of which is expressly incorporated herein by reference in its entirety.

### FIELD OF THE INVENTION

This disclosure relates to compositions and related methods of using and manufacturing the same for control of gene expression and activity of the gene products using nanoparticles functionalized with bioactive molecules including but not limited to peptides, proteins, small RNA molecules such as small interference RNA (siRNA), and longer RNA molecules such as messenger RNA (mRNA).

### BACKGROUND OF THE INVENTION

The ability of cells to normally proliferate, migrate and differentiate to various cell types is critical in embryogenesis and in the function of mature cells, including but not limited to the cells of cardiovascular, immune, intestinal, and brain systems. This functional ability of the cells is altered in various pathological conditions due to acquired or inherited mutations, that may lead to activation of different intracellular signaling pathways and over-expression of various genes (e.g. oncogenes), each of which can contribute to malignant transformation, hyperproliferation and expansion of malignant cells.

Furthermore, hyperproliferation can also be triggered by aberrant loss of expression of some vital genes needed for normal functioning of the cells (e.g. tumor suppressor genes), and such loss of function may lead to malignant transformation and development of different tumors and cancers.

Furthermore, during viral infection, new and/or altered genes (either of viral origin or products of de novo mutations triggered by integration of viral DNA into host cell genome or expressed from the viral RNA) are expressed in the cells and aid with viral replication, which can lead to various severe and sometimes life-threatening complications. Although the immune system frequently is effective in fighting some viral infections, vaccination is a commonly used approach worldwide to prepare organisms to more effectively resist and fight infections. Yet, vaccinations are frequently based on the use of partially or fully inactivated viruses that contain DNA. Every time when exogenous DNA is used with the cells, such DNA integrates into the cell genome and may trigger tumor formation and/or other detrimental consequences. Therefore, non-DNA-based vaccination using proteins or mRNA that preserve the cell genome completely intact represent a much more preferred route of vaccination.

Recent scientific developments present various approaches to control aberrant expression of oncogene-like molecules incorporating administration of exogenous small molecule inhibitors, small interference RNA (siRNA), miRNA, or messenger RNA (mRNA). In addition, viral gene-specific mRNA can be used for vaccination to induce expression of viral gene product and to train cells of the immune system to generate antibody against viruses. Nevertheless, a main barrier to the effective use of siRNA, miRNA, mRNA, and other RNA-based molecules is a lack of highly efficient delivery vehicle capable of transporting siRNA though cell membrane into the cytoplasm of various human cells. The same problem also hinders restoration of gene(s), expression of which is lost during malignant transformation and tumor formation.

Accordingly, despite the advances in the art, a need remains for an efficient approach to deliver biologically active molecules alone or in various combinations into the interior of a cell to effectively induce modulation of gene expression. For example, a need remains to target one or more different abnormal signal transduction pathways and/or induce expression of target gene(s) of interest in various cells while avoiding damage to the chromosomal structure. The present disclosure addresses these and related needs.

### SUMMARY OF THE INVENTION

In some aspects the disclosure provides functionalization and manufacturing methods of linking proteins, peptides, siRNA, microRNA and mRNA to biocompatible nanoparticles for modulating cellular functions. In some aspects, the present disclosure is directed to the multi-functionalized biocompatible nanoparticles themselves. In yet other embodiments, the present disclosure is directed to methods of using the disclosed multi-functionalized biocompatible nanoparticles. In other aspects, the disclosure provides for the multi-functionalized nanoparticles, and compositions, kits, and cells comprising the same.

These and other aspects of the present disclosure will become more readily apparent to those possessing ordinary skill in the art when reference is made to the following detailed description in conjunction with the accompanying drawings.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows two photographs of human primary fibroblasts demonstrating that exemplary multi-functionalized nanoparticles of the present disclosure are highly efficient in cytoplasmic delivery of gene-specific siRNA molecules in primary cells. The left panel shows control human primary fibroblasts treated with the nanoparticles that do not contain siRNA. The right panel shows human primary fibroblasts that were treated with FITC-labeled nanoparticles multi-functionalized with bioactive peptides and siRNA constructs targeting tumor suppressor gene PT10, followed by extensive washes to remove unbound nanoparticles. The cell nuclei were stained with DAPI. The indicated fluorescence (see arrows showing exemplary fluorescence signals) demonstrates presence of PTEN-specific siRNA-functionalized nanoparticles in cell cytoplasm that knocks down the target PT10 gene expression by 60% as determined by qRT-PCR.

FIG. 2 shows two photographs of human primary fibroblasts demonstrating that exemplary multi-functionalized nanoparticles of the present disclosure are highly efficient in delivery and translation of mRNA in the cells. The left panel shows control human primary fibroblast cells exposed to the nanoparticles that do not contain mRNA. The right panel depicts cells treated with NPs functionalized with uncapped mRNA encoding red mCherry protein. mCherry mRNA expression, as determined by red fluorescence (see arrows showing exemplary fluorescence signals) was assessed 26 hours after treatment. The indicated fluorescence confirms that mRNA, including uncapped mRNA delivered by the multi-functionalized nanoparticle can successfully result in efficient translation of the mRNA payload.

### DETAILED DESCRIPTION OF THE INVENTION

The present disclosure is based on the inventor's development of an efficient nanoparticle-based delivery mechanism that effectively facilitates non-integrative delivery of one or more bioactive molecules of various origin, including siRNA, miRNA, mRNA, peptides and proteins into various cell types. The RNA constructs exhibit high functionality and yet preserve intact cell genome. This platform can be multiplexed with multiple bioactive payloads to simultaneously target, e.g., multiple pathways, and provides novel approach for highly efficient regulation of target gene expression. This platform can be applied for numerous applications, including to implement enhanced vaccination, to promote telomere extension, to effectively treat human malignancies, to control expression of genes causing various pathologic conditions, and the like.

In order to deliver biologically active molecules intracellularly, this disclosure provides a universal platform based on a composition including a cell membrane-penetrating nanoparticle with covalently linked biologically active molecules of various origins. To this end, the disclosure presents herein a novel functionalization approach that ensures a covalent linkage of proteins, peptides and different RNAs to nanoparticles. The modified cell-permeable nanoparticles of the present invention provide a universal mechanism for simultaneous intracellular delivery of biologically active molecules for regulation and/or normalization of cellular function, which can be subsequently used in research and development, drug screening and therapeutic applications to improve cellular or organ function and/or body resistance to infections in humans.

In accordance with the foregoing, in one aspect the disclosure provides a composition configured to deliver RNA payloads to the interior of cells. The composition comprises a multi-functionalized nanoparticle core functionalized with:

at least one RNA molecule attached to the solid nanoparticle core by a linker,

at least one positively charged cell penetrating peptide (CPP) attached to the solid nanoparticle core; wherein the functionalized nanoparticle is substantially neutrally charged, negatively or positively charged.

The nanoparticle core is preferably biocompatible, including for example, superparamagnetic iron oxide or gold nanoparticles or polymeric biodegradable nanoparticles similar to those previously described in scientific literature (Lewin M et al., Nat. Biotech. 2000, 18, 410-414; Shen T, et al., Magn. Reson. Med. 1993, 29, 599-604; Weissleder, R. et al. American Journal of Roentgeneology 1989, 152, 167-173). Such nanoparticles can be used, for example, in clinical settings for magnetic resonance imaging (because one of the potential use is to label targeted cells intracellularly and image their routes in vivo as it was done by extracellular labeling of cells and imaging) of bone marrow cells, lymph nodes, spleen and liver (see, e.g., Shen et al., Magn. Reson. Med. 29, 599 (1993); Harisinghani et. al., Am. J. Roentgenol. 172, 1347 (1999)). For example, magnetic iron oxide nanoparticles sized less than 50 nm and containing cross-linked cell membrane-permeable Tat-derived peptide efficiently internalize into hematopoietic and neural progenitor cells in quantities of up to 30 pg of superparamagnetic iron nanoparticles per cell (Lewin et al., Nat. Biotechnol. 18, 410 (2000)). Furthermore, the nanoparticle incorporation does not affect proliferative and differentiation characteristics of bone marrow-derived CD34+ primitive progenitor cells or the cell viability (Maite Lewin et al., Nat. Biotechnol. 18, 410 (2000)). These nanoparticles can be used for in vivo expression of virtually any gene of interest whether the gene expression is lost during oncogenesis or needed for vaccination.

In some embodiments, the nanoparticle core is solid. For example, the solid nanoparticle core can be metallic or non-metallic that include but not limited to chitosan or hydroxyapatite based nanoparticles. Exemplary metallic nanoparticles encompassed by the disclosure include magnetic nanoparticles, and superparamagnetic iron-based, silver, titanium nanoparticles. For example, the nanoparticle core can be or comprise iron (e.g., iron oxide). Another exemplary nanoparticle core is or comprises gold.

In some embodiments the nanoparticle core comprises a biocompatible polymer. For example, the polymer coating such as, e.g., dextran polysaccharide, can have X/Y functional groups, to which functional elements or linkers of various lengths can be attached. The linkers, in turn, are covalently attached to functional groups such as the RNA molecule, and/or optionally the CPP and/or positively charged moiety. The linkers can also be configured to be attached to, e.g., additional proteins, microRNAs and/or peptides (or other small molecules) through their X/Y functional groups. Exemplary functional groups for cross-linking are described in more detail and are encompassed by this aspect of the disclosure.

In some embodiments, the nanoparticle core is an aggregation of polymers without a metal or solid core structure. Instead, the aggregation of polymers encompasses bioactive molecules trapped inside that can be shedding over time leading to long-lasting effects. Such polymeric nanoparticles are known and can be configured by a person of ordinary skill in the art to be multi-functionalized as described herein.

In some embodiments, the nanoparticle core is a solid nanoparticle core that has a size of 50 nm or less in diameter, such as between about 5 nm to about 50 nm in diameter, about 25 to about 45 nm in diameter, about 30 nm to about 45 nm in diameter, about 35 nm to about 45 nm in diameter, about 40 nm to about 45 nm in diameter, about 40 nm to about 50 nm in diameter, about 20 nm to about 30 nm in diameter, or other subranges therein. In exemplary embodiments, the nanoparticle core has a diameter of about 5 nm, about 10 nm, about 20 nm, about 23 nm, about 25 nm, about 28 nm, about 30 nm, about 33 nm, about 35 nm, about 38 nm, about 40 nm, about 45 nm, and about 50 nm.

As indicated above, the nanoparticle-based compositions serve as excellent vehicles for intracellular delivery of biologically active molecules that can be applied, for example, to target intracellular events and modulate cellular function and properties of various cell types of interest. Thus, the composition of this aspect provides nanoparticle-based compositions that multi-functionalized to carry one or more functional payloads. As indicated, the nanoparticle core is functionalized at least with an RNA molecule payload. The RNA molecule can be a short interfering RNA (siRNA), a microRNA (miRNA), or encoding RNA such as messenger RNA (mRNA).

In some embodiments, the RNA molecule is an uncapped mRNA molecule. Messenger RNA (mRNA) generally refers to a single stranded RNA molecule that contains a sequence encoding a peptide or polypeptide of interest. In some embodiments, the mRNA is a “mature” mRNA, meaning that it lacks intron sequences interspersed between encoding exons. mRNA can also have additional modifications that typically occur in eukaryotic cells. For example, the mRNA can have a 5′ cap structure, which comprises an added RNA 7-methylguanosine cap). This is a modified guanine nucleotide that is typically linked through a 5′-5′-triphosphate bond. The 5′ cap structure can canonically preserve stability of the molecule by protecting from degradation by RNAses. Additionally, the mRNA can comprise a polyadenylyl tail at the 3′ end. The “poly(A)” tail also promotes stability of the mRNA by protecting from degradation from exonucleases.

In some embodiments, the mRNA is an uncapped mRNA molecule. As used herein “uncapped” refers to a lack of the canonical 5′ cap structure linked through the 5′-5′-triphosphate bond. In such embodiments, the uncapped 5′ end of the mRNA is bound to a linker, which in turn is bound to the nanoparticle core structure. It was found that the mRNA molecule remains stable if tethered to the nanoparticle at its 5′ end. Without being bound by any particular theory, it is believed that the presence of the nanoparticle with the other functional groups described herein, shield this portion of the mRNA from degradation by the nucleases present in the cells.

In other embodiments, the RNA molecule is a capped mRNA molecule, wherein the 3′ end is of the capped mRNA molecule is covalently bound to the first linker. The first linker is, in turn, bound to the surface of the nanoparticle core.

Regardless of the capped or uncapped configuration, the mRNA molecule can be configured to encode peptides or polypeptides (e.g., functional proteins) of interest according to the vast knowledge of protein and coding sequences known in the art. For example, the mRNA molecule can encode, an antigen of interest, an enzyme of interest (e.g., a telomerase), or detectable marker, among other desirable peptides and polypeptides.

In some embodiments, the composition comprises at least two RNA molecules attached to the solid nanoparticle core. Each of the RNA molecules can be independently attached by linkers, that can be the same or different. The RNA molecules can be the same or different, with at least one of the RNA molecules being an uncapped mRNA molecule with the 5′ end of the uncapped mRNA molecule being covalently bound to the first linker, which is in turn covalently attached to the nanoparticle core.

As indicated, the RNA molecule is attached to the nanoparticle core by a first linker. The linker can be a linear or branching linker. A branching linker is covalently bound to the nanoparticle core through a single point of contact and has a plurality of branches originating at one or more branchpoints. Typically, at least two of the plurality of branches are attached to individual RNA molecules.

In some embodiments, the linker is comprised of one or more linkers each at least 6 ångstroms long, such as at least 6, 7, 8, 9, 10, 11, 12, 13, 14, 15, 16, 17, 18, 19, 20, or more ångstroms long. Without being bound to any particular theory, the distance provided by linkers of this length permit sufficient flexibility and distance from the nanoparticle core to allow ribosomal access to the mRNA payload, thus permitting translation of the mRNA into a peptide or polypeptide product.

In some embodiments, the first linker is a cleavable linker, such as a linker configured to be cleaved within a cell, resulting in release of the payload molecule from the nanoparticle core. Such a release can allow the ribosomal complex formation on the mRNA template to facilitate translation. In some embodiments, the cleavable linker comprises a disulfide bond.

Appropriate linker groups and related methods to attach functional groups to the nanopore are known. See, e.g., U.S. Pat. No. 9,675,708, incorporated herein by reference in its entirety. Illustrative, non-limiting examples of functional linker groups that can be used for crosslinking the RNA molecule (and potentially other functional groups) to the nanoparticle include:

—NH₂ (e.g., lysine, a-NH₂);

—SH;

—COOH;

—NH—C(NH)(NH₂);

carbohydrate;

-hydroxyl (OH); and

attachment via photochemistry of an azido group on the linker.

Illustrative, non-limiting examples of crosslinking reagents include:

SMCC [succinimidyl 4-(N-maleimido-methyl) cyclohexane-1-carboxylate], including sulfo-SMCC, which is the sulfosuccinimidyl derivative for crosslinking amino and thiol groups;

LC-SMCC (Long chain SMCC), including sulfo-LC-SMCC;

SPDP [N-Succinimidyl-3-(pypridyldithio)-proprionate], including sulfo-SPDP, which reacts with amines and provides thiol groups;

LC-SPDP (Long chain SPDP), including sulfo-LC-SPDP;

EDC [1-Ethyl Hydrocholride-3-(3-Dimethylaminopropyl)carbodiimide], which is a reagent used to link a —COOH group with a —NH₂ group;

SM(PEG)n, where n=1, 2, 3, 4 . . . 24 glycol units, including the sulfo-SM(PEG)n derivative;

SPDP(PEG)n where n=1, 2, 3, 4 . . . 12 glycol units, including the sulfo-SPDP(PEG)n derivative;

PEG molecule containing both carboxyl and amine groups; and

PEG molecule containing both carboxyl and sulfhydryl groups.

Illustrative, non-limiting examples of capping and blocking reagents include:

Citraconic Anhydride, which is specific for NH;

Ethyl Maleimide, which is specific for SH; and

Mercaptoethanol, which is specific for maleimide.

The at least one cell-penetrating peptide (CPP) and/or the at least one positively charged moiety can also be attached to the nanoparticle core via linker constructs. Any appropriate linker construct can be used to attach the at least one cell-penetrating peptide (CPP) and/or the at least one positively charged moiety to the nanopore core, such as those described above. In one embodiment, the at least one CPP is attached to the solid nanoparticle core by a second linker. The first linker and the second linker can be the same or different types of linkers. In some embodiments, the first linker and second linker are different, and the second linker is longer than the first linker. In one embodiment, the at least one positively charged moiety is attached to the solid nanoparticle core by a third linker. The first linker and the third linker can be the same or different types of linkers. In one embodiment, the first linker and third linker are different, and the third linker is longer than the first linker. Without being bound by any particular theory, longer lengths in the second and/or third linkers compared to the first linker allows the smaller moieties (i.e., the CPP and/or the positively charged moiety) to be extended further from the surface of the nanopore core notwithstanding the presence of the much bulkier RNA molecule. Arranged as such, the CPP and/or the positively charged moiety can have more opportunity to interact with the surrounding environment.

The at least one positively charged moiety provides further positive charge to offset the negative charge provided by the bulky RNA molecule. There can be multiple units of the same positively charged moiety linked to the same nanoparticle core. Additionally, or alternatively, there can be one or more units of multiple different kinds of charged moieties attached to the same nanoparticle core. In some embodiments, the at least one positively charged moiety is a charged peptide. Exemplary charged peptides can comprise two or more positively charged amino acids, e.g., 2, 3, 4, 5, 6, 7, 8, 9, 10, or more, charged amino acids.

In some embodiments, the solid nanoparticle core has a plurality of mRNA and/or siRNA molecules and a plurality of positively charged moieties linked thereto at a ratio of about 100:1 to about 1:10, for example at a ratio of about 100:1, about 90:1, about 80:1, about 70:1, about 60:1, about 50:1; about 40:1, about 30:1, about 20:1, about 10:1, about 5:1, about 1:1, about 1:5, and about 1:10, and any range therebetween.

As indicated above, the multi-functionalized nanoparticle core is substantially neutrally charged or positively charged. In this regard, the substantial negative charge conferred by the attached mRNA molecule(s) is offset by positive charges. The term “substantially neutral” refers to a near-neutral charge, with slight negative or positive charge tolerated.

Cell penetrating peptides (CPPs) are short peptides that facilitate cellular uptake of the associated construct. In common embodiments, the at least one CPP contains a relatively high abundance of positively charged amino acid (e.g., lysine or arginine) or has alternating patterns of polar, charged amino acids and non-polar, hydrophobic amino acids. In some embodiments, the at least one CPP comprises five to nine basic amino acids. In some embodiments, the at least one CPP comprises five to nine contiguous basic amino acids. Exemplary CPPs include the transactivating transcriptional activator (TAT), obtained from HIV-1, or derivatives thereof, which are encompassed by the present disclosure. See, e.g., Okuyama M., et al., (February 2007). “Small-molecule mimics of an alpha-helix for efficient transport of proteins into cells”. Nature Methods. 4(2): 153-9, incorporated herein by reference in its entirety, for a discussion of other representative CPPs.

In some embodiments, the composition further comprises at least one siRNA molecule attached to the solid nanoparticle core, wherein the at least one siRNA molecule is specific for a gene of interest. As used in this context, the term “specific for” refers to the sequence specificity of the siRNA molecule construct such that it can specifically hybridize to the transcript of a gene of interest, thus interfering with the translation into a functional protein. As such, the siRNA can induce a knock down of functional expression of a target gene of interest. In some embodiments, the composition further comprises two or more different siRNA molecules attached to the solid nanopore core. Each of the two or more siRNA molecules are specific for different genes of interest or are specific for different sequences in a gene of interest. The siRNA molecules and RNA molecules (e.g., mRNA molecules) can be present in a ratio of about 1:20 to about 20:1, such as about 1:20, about 1:15, about 1:10, about 1:5, about 1:1, about 5:1, about 10:1, about 15:1, and about 20:1. In various pathological conditions one or more signaling molecules are aberrantly overexpressed whereas expression of other genes are silenced. Therefore, simultaneous targeting of these molecules with the multi-functionalized nanoparticle to knockdown expression of overexpressed gene(s) by siRNA or induce expression of silenced gene(s) by mRNA or miRNA or other bioactive molecules such as peptides or proteins presents a powerful means to restore normal phenotype.

The disclosure also encompasses embodiments where the multi-functionalized nanoparticle core also comprises other functional molecules, such as proteins, peptides and other small molecules. The additional functional molecules can be rationally selected to further modify or modulate gene transcription or signaling pathways within a cell. See, e.g., U.S. Pat. No. 9,675,708, incorporated herein by reference in its entirety.

The composition can further comprise additional components that facilitate administration to living cells, either in culture or in vivo in a subject. Exemplary components include acceptable carriers, excipients, optional buffering agents, and the like, appropriately formulated for the dose and mode of administration, as known in the art.

In another aspect, the disclosure provides a cell comprising the functionalized nanoparticle described above. In some embodiments, the cells receive an administration of the composition, after which the RNA molecule is expressed into a functional protein that modifies or modulates the cell in some way. In some embodiments, additional optional components such as siRNA constructs further modulate signaling pathways or other gene expression patterns in the cell. The resulting cell can also have therapeutic value upon administration to a subject. Accordingly, the cell can be modified with the administration of the composition described above ex vivo.

In another aspect, the disclosure provides a method of expressing a polypeptide of interest in a cell. The method comprises delivering the composition, as described above, to the cell and permitting expression of the RNA molecule, wherein the RNA molecule encodes the polypeptide of interest. Exemplary, non-limiting polypeptides include antigens, enzymes (e.g., telomerase), and detectable markers. To illustrate, in one embodiment, the composition described above is used as a delivery platform for mRNA encoding a telomerase. Administration of the composition to the cell, either in vitro or in vivo, can result in the efficient delivery and translation of telomerase-encoding mRNA to the cell. The expressed telomerase can elongate the telomeres of the cellular chromosomes, thus promoting a more robust lifespan of the cell. Overall, this can be an integral component of anti-aging treatment in a subject. In another exemplary embodiment, the composition comprises mRNA(s) encoding tumor suppressor gene(s) (TSG), whose normal function is to inhibit cell transformation and malignant clone growth and whose inactivation is advantageous for tumor cell growth, are frequently silenced in a variety of cancers. Exemplary TSGs are PTEN, TP53, p16 and other genes reported as silenced in solid tumor tissues or in blood cancer leukemia (Oliveira A M, Ross J S, Fletcher J A. Tumor suppressor genes in breast cancer: the gatekeepers and the caretakers. Am J Clin Pathol. 2005 December; 124 Suppl:S16-28. doi: 10.1309/5XW3L8LU445QWGQR; Wang L, Wu C, Rajasekaran N, Shin Y: Loss of Tumor Suppressor Gene Function in Human Cancer: An Overview. Cell Physiol Biochem 2018; 51:2647-26930.

The following describes an exemplary approach for assembling the multi-functionalized nanoparticles.

The nanoparticles useful for the disclosed platform can contain a core comprising as an example of iron oxide, hydroxyapatite, or gold, or can be polymeric nanoparticles without a core but containing encapsulated trapped inside bioactive molecules that will be shedding over time and leading to long-lasting effects.

Biocompatible nanoparticles are treated with functional groups (e.g. amine or carboxy groups) on the surface to chemically bind proteins, nucleic acids and short peptides by various means such as described in U.S. Pat. No. 9,675,708, incorporated herein by reference in its entirety. Briefly, the superparamagnetic or alternative nanoparticles can be less than 50 nm or larger in size and 10¹²-10²⁰ nanoparticles per ml with 10 or more amine groups per nanoparticle.

SMCC (from Thermo Fisher) can be dissolved in dimethylformamide (DMF) obtained from ACROS (sealed vial and anhydrous) at the 1 mg/ml concentration. Sample is sealed and used almost immediately.

Ten (10) microliters of the solution are added to nanoparticles in 200 microliter volume. This provided a large excess of SMCC to the available amine groups present, and the reaction is allowed to proceed for one hour. Excess SM and DMF can be removed using an Amicon centrifugal filter column with a cutoff of 3,000 daltons. Five exchanges of volume are generally required to ensure proper buffer exchange. It is important that excess of SMCC be removed at this stage.

Any RNA as described above, and optionally siRNAs, peptide-based molecules, and proteins of interest, are added to the activated nanoparticles. The bioactive molecule-nanoparticle solutions are reacted, and the unreacted molecules are removed by centrifugal filter units with appropriate MW cutoff (in case of GFP protein it is 50,000 dalton cut-off). The sample is stored at −80° C. freezer or at 4° C. Instead of using Amicon spin filter columns, small spin columns containing solid size filtering components, such as Bio Rad P size exclusion columns can also be used. It should also be noted that SMCC also can be purchased as a sulfo derivative (Sulfo-SMCC), making it more water soluble. DMSO may also be substituted for DMF as the solvent carrier for the labeling reagent; again, it should be anhydrous.

It is known that RNA molecules are negatively charged, and as such, they do not easily penetrate through cell membrane. As described, herein a combination approach provides a balance of positively charged peptides and negatively charged RNA molecules. Furthermore, while siRNA/miRNA molecules are usually short, usually less than 50 nucleotides in length, and hence, their cumulative negative charge is relatively small, the gene-specific mRNAs are much longer (usually more than 500 nucleotides in length, and more frequently more than 1500 nucleotides) with a substantially larger cumulative negative charge. Therefore, to deliver such RNA molecules intracellularly, a certain ratio of positively charged molecules, such as peptides composing of at 2 or more positively charges amino acids is required to ensure penetration of a nanoparticle multi-functionalized with such peptide and one or more molecules of mRNA.

Nucleic acids can be attached to the nanoparticles either at the 5′ or 3′ end. Uncapped mRNA molecules can be attached by the 5′ end to the linker, as described above. Alternatively, T4 RNA ligase can be used to add a nucleotide with a sulfur group to the 3′ end of an RNA molecule, which can then be used to attach to the nanoparticle.

An exemplary protocol for 3′ end labeling comprises combining the following in a single RNase-free microfuge tube:

2 μl 10×T4 RNA Ligase Buffer

50-100 pmol RNA

equimolar amount (50-100 pmol) [³²P]pCp

RNase-free water to a final volume of 18 μl

Add 2 μl T4 RNA Ligase (10 U);

incubating at 4 C overnight (10-12 hours); and remove unincorporated label by applying the mixture to an RNase-free Sephadex G-25 or G-50 spin column (e.g., NucAway Spin Columns) following the manufacturer's recommendations.

All the other crosslinking reagents can be applied in a similar fashion. SPDP is also applied to the protein/applicable peptide in the same manner as SMCC. It is readily soluble in DMF. The dithiol is severed by a reaction with DTT for an hour or more. After removal of byproducts and unreacted material, it is purified by use of an Amicon centrifugal filter column with 3,000 MW cutoff.

Another approach for labeling a nanoparticle with a peptide, different RNA molecules or protein can be to use two different bifunctional coupling reagents, as described in U.S. Pat. No. 9,675,708, incorporated herein by reference in its entirety.

In one embodiment, various ratios of SMCC labeled proteins and peptides are added to the beads and allowed to react. The linker provides conformational flexibility to the attached molecules so they can rotate and bind interacting partners. Furthermore, the linker can cleavable, more specifically, likely to be cleaved by an intracellular protease or reduced by intracellular molecules, so as to separate the NP from the bioactive molecules once in the cell.

In another aspect, the present invention is also directed to a method of delivering simultaneously several bioactive molecules (e.g. more than one siRNA specific to different genes of interest alone or in combination with gene-specific mRNAs) attached to functionalized nanoparticles for modulation of intracellular activity aimed at knocking down expression of oncogenes or other genes known to trigger or mediate tumorigenesis or lead to expansion of malignant tumor cells, and/or aimed at inducing expression of gene product lost during tumorigenesis or required for robust immune response and protective against various infections. For example, human cells, fibroblasts or other cell types that are either commercially available or obtained using standard or modified experimental procedures are first plated under sterile conditions on a solid surface with or without a substrate to which the cells adhere (feeder cells, gelatin, martigel, fibronectin, and the like). The plated cells are cultured for a time with a specific factor combination that allows cell division/proliferation or maintenance of acceptable cell viability. Examples are serum and/or various growth factors as appropriate for the cell-type, which can later be withdrawn or refreshed, and the cultures continued. The plated cells are cultured in the presence of functionalized biocompatible cell-permeable nanoparticles with covalently linked cardiac-specific reprogramming factors attached using various methods briefly described herein and elsewhere (see, e.g., US 2014/0342004, incorporated herein by reference in its entirety) in the presence or absence of magnetic field. The use of a magnet in case of superparamagnetic nanoparticles renders an important increase in the contact surface area between the cells and nanoparticles and thereby reinforces further improved penetration of functionalized nanoparticles through the cell membrane. When necessary, the cell population is treated repeatedly with the functionalized nanoparticles to deliver the bioactive molecules intracellularly.

The cells are maintained attached or suspended in culture medium, and non-incorporated nanoparticles are removed by centrifugation or cell separation, leaving cells that are present as clusters. The cells are then resuspended and recultured in fresh medium for a suitable period. The cells can be taken through multiple cycles of separating, resuspending, and reculturing, until alterations in targeted specific bioactive molecules and/or signaling pathways are observed. The current invention is applicable to a broad range of cell types can be used such as human fibroblasts, blood cells, epithelial cells, mesenchymal cells, etc.

Furthermore, these multi-functionalized nanoparticles can also be introduced either directly or through catheter-mediated delivery or directly into the tumor (to treat cancer) or into other tissue (e.g. intramuscularly) for vaccination purposes.

Regulation of cell activity, whether direct or indirect, is based on the treatment of various cell types or tissues with bioactive molecules that may include various proteins, peptides, small molecules, microRNA, siRNA, mRNA, etc. These bioactive molecules may not penetrate through cell membrane and may not reach the cell nuclei without a special delivery vehicle. Furthermore, these bioactive molecules alone have short half-life and undergo degradation upon exposure to various proteases and nucleases. These disadvantages result in reduced efficacy of the bioactive molecules and require much higher or repeated doses of treatment to achieve a noticeable effect, if any. Therefore, in the current invention functionalized nanoparticles are used to overcome the abovementioned disadvantages. More specifically, these bioactive molecules when linked to the nanoparticles at various ratios and compared with the original “naked” state, acquire new physical, chemical, biological functional properties, that confer cell-penetrating and intracellular activity targeting ability, resulting in improved resistance to premature degradation, and the acquired capability to simultaneously regulate and control the expression of several target genes of interest and/or intracellular signal transduction pathways.

In one aspect, the disclosure provides methods and compositions for increasing the telomeres in a cell. Every time a cell divides, the chromosomal telomere is shortened. After a number of rounds of division, e.g., about 40, the telomeres can be so shortened that it affects the viability and health of the cell, leading to an aged phenotype and ultimately cell death. At a tissue or organismal level, this manifests in aging and lower biological activity. Viral-based research has demonstrated that the delivery of exogenous nucleic acids encoding telomerases to cells can result in expression of telomerase enzymes. See, Ojeda, Diego, et al. “Increased in vitro glial fibrillary acidic protein expression, telomerase activity, and telomere length after productive human immunodeficiency virus-1 infection in murine astrocytes.” Journal of Neuroscience Research 92.2 (2014): 267-274; and Hong, Jin Woo, and Chae-Ok Yun. “Telomere Gene Therapy: Polarizing Therapeutic Goals for Treatment of Various Diseases.” Cells 8.5 (2019): 392, each of which is incorporated herein by reference in its entirety. The expressed telomerases lengthen the target cells' telomeres resulting in a younger cell phenotype, and increased longevity of the cell and the associated tissue. As indicated above, the disclosed nanoparticles provide alternative cell-transformation vehicles that, when functionalized with mRNA, provide an efficient alternative to viral-based expression of heterologous genes. Moreover, the presently disclosed nanoparticle-based approach has the additional benefit of long-term stability and avoids integrating foreign molecules into the chromosomes and, thus, avoids potentially deleterious side-effects of viral-based gene therapies. Therefore, telomerase-based treatments that are implemented via multi-functionalized nanoparticles as described herein will successfully cause expression of telomerases in target cells, and achieve at least equivalent results in promoting a young phenotype and extend longevity in the cell, without risk of interruptive and deleterious chromosome insertions.

This aspect can be applied in method of treatment or therapy whereby a target cell, cells, tissues, etc. in vivo are contacted with a therapeutically effective amount of the disclosed nanoparticle functionalized with mRNA encoding a telomerase. The method can be applied to generally extend telomerases in the cell/tissue/body thereby promoting a younger phenotype, increased longevity, and reduce or reverse the effects of aging. Administration can be systemic, or local. In some embodiments, where the target cells or tissues are in the central nervous system, the administration can be, e.g., via intra-thecal injection.

Another use of the delivery platform encompassed herein is the screening/testing of a bioactive molecule (compound or combination of two more compounds) for an effect on cell activity. This involves combining the compounds attached to the multi-functionalized nanoparticle using methods disclosed herein with a cell population of interest (e.g. fibroblasts, blood cells, mesenchymal cells) or upon tissue injection, culturing/incubating for suitable period and then determining any modulatory effect resulting from the activities of the compound(s) delivered with multi-functionalized nanoparticles.

Another use of the delivery platform encompassed herein is the formulation of specialized cells as a medicament or in a delivery device intended for treatment of a human or animal body. This enables the clinician to administer the functionalized nanoparticles in or around the normal or abnormal tissue of interest either from the vasculature or directly into the muscle or organ wall, thereby allowing the bioactive molecules used to enter the cells and control the damage, and participate in regeneration/regrowth of the tissue's musculature and restoration of specialized function.

### Additional Definitions

Unless specifically defined herein, all terms used herein have the same meaning as they would to one skilled in the art of the present invention. Practitioners are particularly directed to Sambrook J., et al. (eds.) Molecular Cloning: A Laboratory Manual, 3rd ed., Cold Spring Harbor Press, Plainsview, N.Y. (2001); Ausubel F. M., et al. (eds.), Current Protocols in Molecular Biology, John Wiley & Sons, New York (2010); and Coligan J. E., et al. (eds.), Current Protocols in Immunology, John Wiley & Sons, New York (2010) for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The terms “subject,” “individual,” and “patient” are used interchangeably herein to refer to a mammal being assessed for treatment and/or being treated. In certain embodiments, the mammal is a human. The terms “subject,” “individual,” and “patient” encompass, without limitation, individuals having cancer. While subjects may be human, the term also encompasses other mammals, particularly those mammals useful as laboratory models for human disease, e.g., mouse, rat, dog, non-human primate, and the like.

The term “treating” and grammatical variants thereof may refer to any indicia of success in the treatment or amelioration or prevention of a disease or condition (e.g., a cancer, infectious disease, or autoimmune disease), including any objective or subjective parameter such as abatement; remission; diminishing of symptoms or making the disease condition more tolerable to the patient; slowing in the rate of degeneration or decline; or making the final point of degeneration less debilitating.

The treatment or amelioration of symptoms can be based on objective or subjective parameters; including the results of an examination by a physician. Accordingly, the term “treating” includes the administration of the compounds or agents of the present disclosure to prevent or delay, to alleviate, or to arrest or inhibit development of the symptoms or conditions associated with disease or condition (e.g., a cancer, infectious disease, or autoimmune disease). The term “therapeutic effect” refers to the reduction, elimination, or prevention of the disease or condition, symptoms of the disease or condition, or side effects of the disease or condition in the subject.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

This example describes an assay where multi-functionalized nanoparticles encompassed by the present disclosure were used to successfully deliver siRNA payloads that were able to measurably reduce functional expression of the target gene.

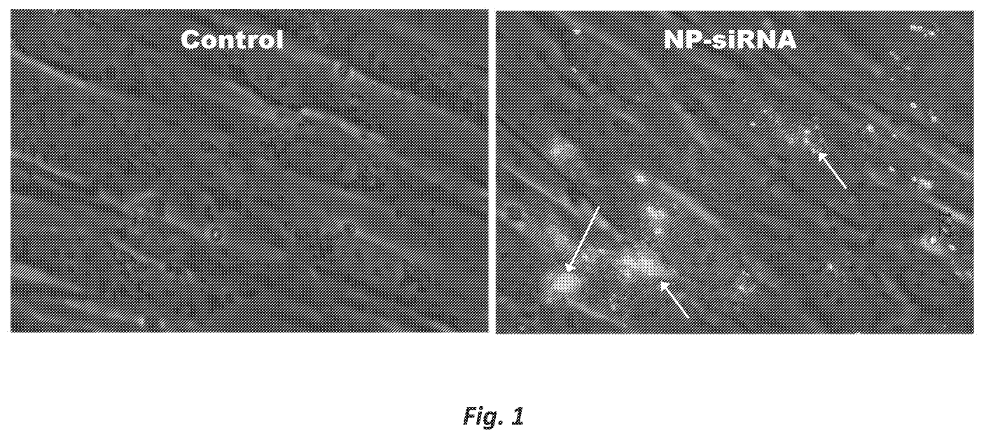

A cell penetrant nanoparticle with free amine group available on its surface is treated with a bi-specific linker capable of forming a covalent bond with the amine group on the nanoparticle resulting in a linker-functionalized nanoparticle. Human PTEN siRNA chemically modified to bind the free end of the nanoparticle linker containing for example maleimide is added to form a covalent bond that is followed by extensive wash or other means of separation from unbound PTEN siRNA molecules. The resultant nanoparticle when added to the cells expressing PTEN gene is delivering its PTEN-specific siRNA cargo into the cell cytoplasm as depicted in FIG. 1. Once in the cytoplasm of the cells, the siRNA molecules interact with PTEN mRNA and trigger a multistep process known as mRNA degradation that results in reduction in the PTEN gene expression. Using this approach, various degree of PTEN knockdown can be achieved, The PTEN-multi-functionalized nanoparticles generated as described above are capable of knocking down at least 60% of the normal PTEN expression level as determined by quantitative real time RT-PCR using PTEN-specific primers and RNA isolated from siRNA-treated cells and control cells treated with nanoparticles in the absence of siRNA.

These data establish a proof of concept that the disclosed multi-functionalized nanoparticles can efficiently deliver RNA-based payloads into the interior of the cell with functional roles intact. More specifically, the data demonstrate that the multi-functionalized nanoparticles can deliver siRNA constructs to successfully knock down expression of a target gene of interest.

### Example 2

This example describes an assay where the multi-functionalized nanoparticles encompassed by the present disclosure were used to successfully deliver an mRNA, which was efficiently expressed by the cellular translation machinery to result in measurable gene expression.

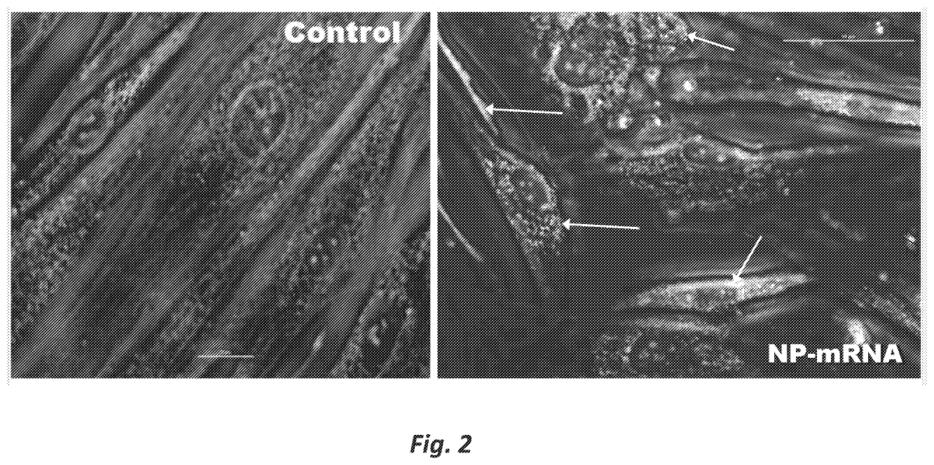

In this case, the mCherry mRNA that upon translation expresses a red fluorescent protein is generated using mCherry cDNA cloned under control of T7 promoter. The T7 in vitro transcription kit (New England Biolabs, Ipswich, Mass.) is used to generate an uncapped mCherry mRNA, which is subsequently purified using Qiagen RNA purification columns (Qiagen, Germantown, Md.), chemically modified using alkaline phosphatase and S-gamma-ATP according to the manufacturer's instructions (New England BioLabs) and reacted with the linker-functionalized cell-penetrant nanoparticle generated as described in Example 1 above. The resultant mRNA multi-functionalized nanoparticles or the nanoparticles in the absence of mCherry mRNA are added to the cells and the cells are cultured in CO2 incubator. The FIG. 2 depicts fluorescent microscopy of normal cells treated with control nanoparticles lacking mRNA that show normal human cells with virtually no red fluorescence indicating a lack of red mCherry protein expression. In contrast, the cells treated with mCherry mRNA-multi-functionalized nanoparticles demonstrate a distinct red staining (arrows to the white punctate areas in the right panel) that indicate a successful expression of red mCherry protein distributed throughout the cell cytoplasm.

These data establish a proof of concept that the disclosed multi-functionalized nanoparticles can efficiently deliver mRNA-based payloads into the interior of the cell with functional roles intact. More specifically, the data demonstrate that the multi-functionalized nanoparticles can deliver mRNA molecules into the cell cytoplasm where the mRNA is successfully translated resulting in the expression of the target gene of interest. It is also emphasized that the preparation of these non-integrating functionalized nanoparticles does not involve any DNA molecules that could integrate into the cell genome and disrupt normal gene expression pattern.

These data establish a proof of concept that the disclosed multi-functionalized nanoparticles can efficiently deliver encoding mRNA constructs leading to efficient expression of a target gene of interest.

### Example 3

Using the siRNA and mRNA multi-functionalization approach described in Examples 1 and 2 above, the non-integrating nanoparticles functionalized with a positively charged peptide, a set of signal transduction molecule-specific siRNAs (capable of targeting beta-catenin, mTOR and Raf1 mRNAs) that are overexpressed in various human tumors can be generated. Briefly, the human cancer cell lines or tumors are treated with functionalized nanoparticles once or repeatedly (2 or more times), which results in delivery of these bioactive molecules to the cytoplasm of the treated cells or tissue and knockdown in expression from these target gene mRNAs. The outcome of such simultaneous regulation (inhibition) of several aberrant in cancer signal transduction pathways is monitored using various molecular biology, biochemistry and cell biology techniques. Specifically, expression of targeted genes can be determined by RNA isolation followed by reverse transcribed PCR (RT-PCR) or real-time quantitative qRT-PCR, immunostaining of the cells using appropriate antibodies, or by flow cytometry analyses of the cultured cells. These gene-specific targeting using siRNA-multi-functionalized nanoparticles can inhibit abnormal signaling that contributes to malignant cell growth and thereby restoring normal phenotype.

### Example 4

Nanoparticles multi-functionalized with a set of gene-specific siRNAs (like the one mentioned above) and with TP53 tumor suppressor-specific mRNA (expression of TP53 is known to be lost in many human cancers) are used to deliver these molecules into the cancer cells or directly into the tumor with silenced TP53 expression. This combination of bioactive siRNA and mRNA molecules simultaneously introduced into the cells as generated and described in Examples 1-3 above is a novel and powerful way to inhibit aberrant signaling pathways and restore the expression of silenced P53 gene, resulting in cell growth reduction or elimination of tumor cells.

The preparation of these non-integrating functionalized nanoparticles does not involve any DNA molecules that could integrate into the cell genome and disrupt normal gene expression pattern. The present invention may be embodied in other specific forms without departing from the spirit or essential characteristics thereof.

### Example 5

For vaccination, the nanoparticle is functionalized with a combination of positively charged cell-penetrating peptide and gene-specific mRNA molecules. Direct treatment of human cells or direct intramuscular, intravenous, intranasal or over the skin administration of these multi-functionalized cell-penetrant nanoparticles results in highly efficient delivery of the mRNAs into the cell cytoplasm, followed by translation of the mRNA and generation of the antigen need to trigger appropriate immune response or other desired effects. Additional gene-specific mRNAs alone or in combination with other types of molecules (e.g. proteins) may also be functionalized onto the nanoparticles if needed using the similar functionalization routes described above to accentuate the expression from delivered mRNAs and immunogenic response.

While illustrative embodiments have been illustrated and described, it will be appreciated that various changes can be made therein without departing from the spirit and scope of the invention.

The embodiments of the invention in which an exclusive property or privilege is claimed are defined as follows:

1. A composition, comprising:
a solid nanoparticle core functionalized with:
at least one RNA molecule attached to the solid nanoparticle core by a first linker, at least one cell penetrating peptide (CPP) attached to the solid nanoparticle core; and
at least one positively charged moiety linked to the solid nanoparticle core;
wherein the functionalized nanoparticle is substantially neutrally charged, negatively or positively charged.

2. The composition of claim 1, wherein the solid nanoparticle core is metallic or non-metallic.

3. The composition of claim 1 or claim 2, wherein the nanoparticle core is superparamagnetic.

4. The composition of claim 2, wherein the solid nanoparticle core comprises iron, gold, or other metals as described above.

5. The composition of one of claims 1-4, wherein the solid nanoparticle core has a size of 50 nm or less in diameter.

6. The composition of claim 1, wherein the RNA molecule is an uncapped mRNA molecule with a 5′ end and a 3′ end, wherein the 5′ end of the uncapped mRNA molecule is covalently bound to the first linker.

7. The composition of claim 1, wherein the RNA molecule is a capped mRNA molecule with a 5′ end and a 3′ end, wherein the 3′ end is of the capped mRNA molecule is covalently bound to the first linker.

8. The composition of claim 6 or claim 7, wherein the mRNA molecule is at least about 150 nucleotides in length.

9. The composition of claim 6 or claim 7, wherein the mRNA molecule encodes an antigen of interest.

10. The composition of claim 6 or claim 7, wherein the mRNA molecule encodes an enzyme of interest.

11. The composition of claim 10, wherein the mRNA molecule encodes a telomerase.

12. The composition of claim 6 or claim 7, wherein the mRNA molecule encodes the detectable protein marker.

13. The composition of claim 1, wherein the first linker is a linear linker.

14. The composition of claim 1, wherein the first linker is a branching linker with a single point of contact to the solid nanoparticle core and a plurality of branches, wherein at least two of the plurality of branches are attached to individual RNA molecules.

15. The composition of claim 1, wherein the first linker is at least 6 ångstroms long.

16. The composition of claim 1, wherein the first linker is a cleavable linker.

17. The composition of claim 16, wherein the cleavable linker is configured to be cleaved within a cell.

18. The composition of claim 16 or claim 17, wherein the cleavable linker comprises a disulfide bond.

19. The composition of one of claims 16-18, wherein the cleavable linker is acid labile or other types of linker.

20. The composition of claim 1, wherein the at least one CPP is attached to the solid nanoparticle core by a second linker, wherein the first linker and the second linker are the same or different.

21. The composition of claim 20, wherein the first linker and second linker are different, and the second linker is longer than the first linker.

22. The composition of claim 1, wherein the at least one positively charged moiety is attached to the solid nanoparticle core by a third linker, wherein the first linker and the third linker are the same or different.

23. The composition of claim 22, wherein the first linker and third linker are different, and the third linker is longer than the first linker.

24. The composition of claim 1, when the at least one positively charged moiety is a charged peptide.

25. The composition of claim 24, wherein the charged peptide contains two or more positively charged amino acids.

26. The composition of any preceding claim, wherein the solid nanoparticle core has a plurality of mRNA or siRNA molecules and a plurality of positively charged moieties attached thereto at a ratio of about 100:1 to about 1:10.

27. The composition of claim 1, wherein the composition comprises at least two mRNA molecules attached to the solid nanoparticle core, wherein the mRNA molecules can be the same or different, and wherein at least one of the mRNA molecules is an uncapped mRNA molecule with a 5′ end and a 3′ end, wherein the 5′ end of the uncapped mRNA molecule is covalently bound to the first linker.

28. The composition of claim 1, wherein the at least one CPP comprises five to nine basic amino acids.

29. The composition of claim 28, wherein the at least one CPP comprises five to nine contiguous basic amino acids.

30. The composition of claim 1, further comprising at least one siRNA molecule attached to the solid nanoparticle core, wherein the at least one siRNA molecule is specific for a gene of interest.

31. The composition of claim 30, further comprising a two or more different siRNA molecules attached to the solid nanopore core, wherein each of the two or more siRNA molecules are specific to different genes of interest or different sequences in a gene of interest.

32. The composition of claim 30 or claim 31, wherein the siRNA molecules and RNA molecules are present in a ratio of about 1:20 to about 20:1.

33. A cell comprising the functionalized nanoparticle recited in one of claims 1-32.

34. A method of expressing a polypeptide of interest in a cell, comprising delivering the composition as recited in any of claims 1-32 to the cell and permitting expression of the RNA molecule, wherein the RNA molecule encodes the polypeptide of interest.

35. The method of claim 34, wherein the polypeptide is an antigen.

36. The method of claim 34, wherein the polypeptide is an enzyme.

37. The method of claim 36, wherein the enzyme is a telomerase.

38. The method of claim 34, wherein the polypeptide is a detectable marker or a structural protein.
